(19) United States
(12) Patent Application Publication
(10) Pub. No.: US 2025/0177594 A1
(43) Pub. Date: Jun. 5, 2025

(54) DISINFECTION CONFIRMATION DEVICE

(71) Applicant: CareFusion 303, Inc., San Diego, CA (US)

(72) Inventors: Vivek PANDEY, Bangalore (IN); Chetan Satish Dhuri, Bangalore (IN); Krishan Yadavendra, Agra (IN); Leandro Forciniti, Wharton, NJ (US)

(21) Appl. No.: 18/525,155

(22) Filed: Nov. 30, 2023

(51) Int. Cl. A61L 2/28; A61L 2/18; A61M 39/16

(52) U.S. Cl. CPC A61L 2/28; A61L 2/18; A61M 39/162; A61L 2202/123; A61L 2202/24; A61M 2039/167

(57) ABSTRACT

A device for obtaining confirmation of completion of disinfection includes an electrically activated light source. The device further includes at least one enclosure having walls formed of a diffusion control membrane and enclosing a salt, which is soluble in a disinfectant used for the disinfection. The at least one enclosure and the light source are electrically connected in series.

### TECHNICAL FIELD

The present disclosure generally relates to components for medical infusion apparatus, in particular, to a device for enabling confirmation of disinfection of components for medical infusion apparatus.

### BACKGROUND

When infusion tubing is connected or changed during treatment of a patient, various components that are exposed to air, such as e.g., connectors, need to be disinfected to avoid blood stream infection. Infection Nursing Society (INS) has prescribed a standard operating procedure for disinfecting such components, which requires disinfection of the components for a certain amount of time. However, research suggests that healthcare providers do not always disinfect the components for the prescribed amount of time. This is likely because the providers do not have an accurate way to measure whether the component has been disinfected.

### SUMMARY

In view of the challenges described above, a continuing need exists for a means that can provide a visual confirmation that the component has been properly disinfected are desired. Aspects of the subject technology relate to devices and methods that enable visual confirmation that a component such as a connector has been suitably disinfected. The devices and methods can be suitably configured to provide the confirmation for a variety of disinfectants and for use on various types of components including, for example, needle-free connectors, used for connecting medical infusion tubing. The devices described herein can be used as standalone devices that can be coupled to different components or integrated into a component during manufacturing of the component.

Advantages of the subject technology include improved patient safety and workflow efficiency for healthcare providers by providing an easy and in-built method for obtaining confirmation of adequate disinfection of the components used in medical infusion apparatus prior to use of the components. For example, in an aspect of the present disclosure, a device for obtaining confirmation of completion of disinfection, that includes an electrically activated light source, and at least one enclosure having walls formed of a diffusion control membrane and enclosing a salt. The salt is selected to be soluble in a disinfectant used for the disinfection. The at least one enclosure, the power supply and the light source are electrically connected in series. The device may additionally include a power source for providing electrical power to the at least one light source. Advantageously, by suitably selecting the salt the device can be modified to obtain for confirmation of disinfection when using a variety of different disinfectants. In addition, the device can also provide a visual indication that the disinfectant has evaporated and the component is ready for use.

Another example includes a needle-free connector having a power supply, an electrically activated light source and at least one enclosure having walls formed of a diffusion control membrane and enclosing a salt. The salt is selected to be soluble in a disinfectant used for the disinfection. The at least one enclosure, the power supply and the light source are electrically connected in series. The needle-free connector may be suitably modified to provide a visual indication of completion of disinfection using a variety of disinfectants by selecting a suitable salt that is soluble in the particular disinfectant being used.

Embodiments include one or more of the following features individually or combined. For example, the diffusion control membrane is configured to control permeation of the disinfectant through the diffusion control membrane such that ionic mobility of the salt dissolved in the disinfectant is sufficient to enable activation of the light source only after the disinfectant has permeated through the diffusion control membrane for a predetermined amount of time. Such diffusion control membrane may include a material such as, for example, polyethersulfone (PES), polystyrene, molybdenum disulfide, or graphene.

Additional advantages of the subject technology will become readily apparent to those skilled in this art from the following detailed description, wherein only certain aspects of the subject technology are shown and described, simply by way of illustration. As will be realized, the subject technology is capable of other and different configurations, and its several details are capable of modifications in various other respects, all without departing from the subject technology. Accordingly, the drawings and description are to be regarded as illustrative in nature, and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A illustrates a device for providing confirmation of completion of disinfection, in accordance with aspects of the present disclosure.

FIG. 1B is a schematic representation of an enclosure enclosing a salt, in accordance with aspects of the present disclosure.

FIG. 2 illustrates a needle-free connector that can provide confirmation of completion of disinfection, in accordance with aspects of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Aspects of the subject technology relate to components of medical infusion apparatus that need to be disinfected before use to avoid risk of blood stream infections. Infusion nursing society (INS) provides a standard operating procedure for disinfecting such components. The procedure includes scrubbing the component for a certain amount of time with a disinfectant swab before the component is used. The amount of time for which the component is to be scrubbed can vary depending on the specific disinfectant. The prescribed amount of time is based on empirical data collected during studies conducted to determine the amount of time needed to obtain a certain amount (e.g., 99.99%) disinfection of the component using the particular disinfectant with a prescribed method of scrubbing the component with the disinfectant. For example, the INS recommends scrubbing a needle-free connector for at least 15 seconds with an isopropyl alcohol swab (70% solution).

However, during use, it is often difficult for the healthcare provider (e.g., an infusion nurse) to keep track of the time, and consequently, the provider may not know whether the component is properly disinfected. Indeed, research indicates that the providers often fail to scrub the components for the prescribed amount of time. Additionally, it is also difficult for the provider to determine when the component is ready for use, i.e., that the disinfectant has evaporated or is no longer present on the surface of the component.

Aspects of the subject technology relate to devices and methods that facilitate proper disinfection of components of infusion tubing by providing a visual confirmation that the disinfection has been completed according to prescribed procedures. In addition, the devices and methods disclosed herein provide a visual indication that the disinfectant has evaporated from the component and that the component is ready for use. Advantageously, the device may be attachable to existing components or may be integrated within the components at the time of manufacturing the components so as to eliminate additional steps during the workflow. Various aspects of the present disclosure provide a medical grade needle-free connector that has an in-built mechanism for providing visual confirmation that the needle-free connector is properly disinfected, and further visual confirmation that the disinfectant used for disinfection has evaporated and the connector is ready for use.

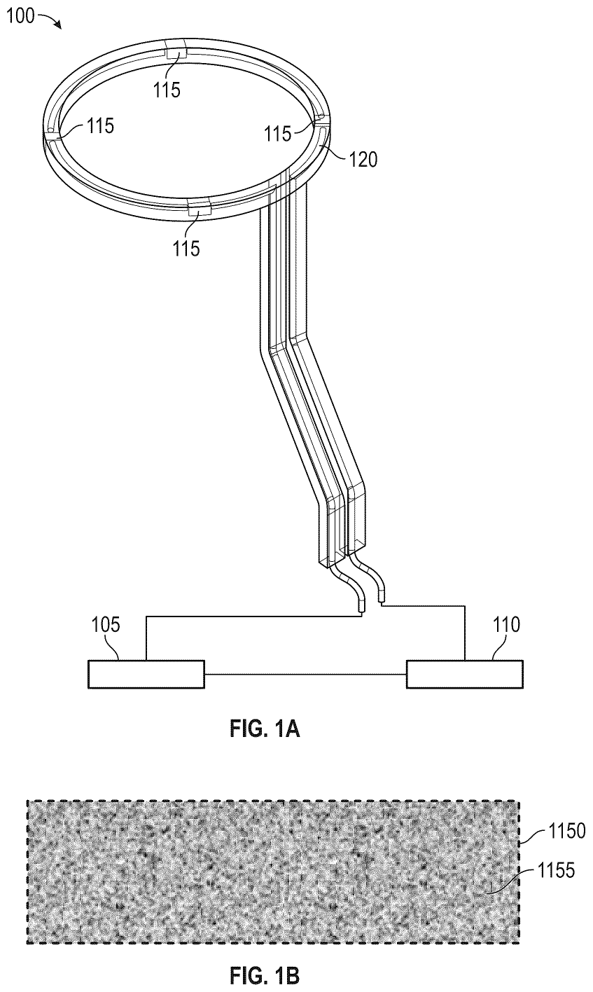

FIG. 1A illustrates a schematic of a device for providing visual confirmation of completion of disinfection, in accordance with aspects of the present disclosure.

In some embodiments, the device 100 may include a power supply 105, an electrically activated light source 110, and at least one enclosure 115. The power supply 105, the light source 110 and the at least one enclosure 115 are connected in series by conducting leads 120.

The device 100, as shown in FIG. 1, may be designed to fit snugly on an outer rim of a needle-free connector used for connecting medical infusion apparatus. However, the shape and size of the device is not particularly limited.

The power supply 105 may be any suitable power supply sufficient to provide electrical power to the electrical circuit formed by the light source 110, the at least one enclosure 105 and the wires 120. Thus, the power supply 105 may be a commercially available battery. While the battery capacity is not particularly limited, in some embodiments, depending on the design of the device, the battery size may have to be limited, thereby limiting the battery capacity. However, those of ordinary skill in the art, upon understanding the present disclosure, would be able readily select a suitable battery for the device.

The light source 110 may be any suitable light source. However, based on the specific application of the device 100, a light emitting diode (LED) may be used as the light source 110 in some embodiments. While any suitable commercially available LED that can fit the physical design parameters of the device may be used, those of ordinary skill in the art would recognize that the selected LED should have sufficient luminous intensity to distinguish between the ON and OFF states of the LED at least in diffused indoor lighting. Thus, for example, a suitable LED may have a luminous intensity of at least around 10 med. Suitable form factor for the LED may depend on design parameters of device 100.

The conducting leads 120 may be standard insulated (or laminated) copper wires used for connecting electrical components. However, there is no particular limitation to the type of conducting leads 120 that can be used in the device so long as the wires allow the device 100 to be formed within the desired design parameters.

FIG. 1B shows a schematic of the enclosure 115. At least one of the walls 1150 of the enclosure 115 comprises a diffusion control membrane. The enclosure 115 encloses an ionic salt 1155. The ionic salt 1155 is selected to be soluble in a disinfectant. The diffusion control membrane is selected to allow diffusion of a disinfectant such as, for example, isopropyl alcohol through the walls, but block the diffusion of the ionic salt 1155 and/or the ions of the salt once the salt is dissolved in the disinfectant.

The size and shape of the enclosure 115 is selected to allow diffusion of the disinfectant through the walls 1150 at a rate that enables a gradual increase in ionic mobility of the salt to allow sufficient current to flow through the electrical circuit to activate the light source 110 only after a prescribed amount of time. Thus, the enclosure 115 functions as a variable resistor in the electrical circuit and the electrical resistance provided by the enclosure 115 is reduced as more disinfectant diffuses through the walls 1150. When the resistance offered by the enclosure 115 is reduced to a threshold value, sufficient current flows through the electrical circuit to activate the light source 110, thereby providing a visual confirmation that the disinfection is complete.

Thus, in some embodiments, the volume of the enclosure 115 may range from about 1 nl to about 1 μl. For example, the enclosure 115 may have a volume of about 1 nl, about 5 nl, about 10 nl, about 15 nl, about 20 nl, about 25 nl, about 30 nl, about 35 nl, about 40 nl, about 45 nl, about 50 nl, about 55 nl, about 60 nl, about 65 nl, about 70 nl, about 75 nl, about 80 nl, about 85 nl, about 90 nl, about 100 nl, about 110 nl, about 120 nl, about 130 nl, about 140 nl, about 150 nl, about 160 nl, about 170 nl, about 180 nl, about 190 nl, about 200 nl, about 220 nl, about 240 nl, about 260 nl, about 280 nl, about 300 nl, about 325 nl, about 350 nl, about 375 nl, about 400 nl, about 450 nl, about 500 nl, about 550 nl, about 600 nl, about 650 nl, about 700 nl, about 750 nl, about 800 nl, about 850 nl, about 900 nl, about 950 nl, about 1000 nl, or any volume between any two of these volumes.

The diffusion control membrane is selected not only to control the material that can diffuse through the membrane, but also to control the rate of diffusion. The diffusion control membrane controls the rate of diffusion of the disinfectant such that the resistance offered by the enclosure 115 is reduced to the threshold value only after a prescribed amount of time. Thus, only after the disinfectant is applied to the device for the prescribed amount of time will the light source 110 be activated, thereby providing a visual confirmation that the device has been exposed to the disinfectant for the prescribed amount of time and the disinfection process has been properly performed.

In some embodiments, a certain amount of salt 1155 may be enclosed in the enclosure 115, and the enclosure is sealed with the diffusion control membrane 1150. The salt 1155 is selected to be dissolvable in the disinfectant that will be used for disinfecting the components. Examples of salts that can be used include, but are not limited to, inorganic salts such as potassium chloride, sodium chloride, and magnesium chloride, or organic salts such as caprylic acid, and cetyltrimethylammonium bromide (CTAB).

In some embodiments, the enclosure 115 and the salt 1155 therein are designed and configured to enable reduction of the resistance to the threshold value after a time period of ranging from about 1 second to about 1 minute. In other words, the enclosure and the salt therein are designed and configured such that the light source is activated only after the disinfectant diffuses through the walls 1150 for a time ranging from about 1 second to about 1 minute. Thus, in some embodiments, the device 100 is designed and configured such that the light source is activated after the exposure to the disinfectant for about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 60 seconds, or even longer.

Additionally, when the disinfectant evaporates, the ionic mobility of the salt decreases, thereby increasing the resistance of the salt to above the threshold value, thereby deactivating the light source 110 and providing an indication that disinfectant has evaporated and the component is ready for use.

The resistance offered by the enclosure may thus, range from several kiloohms in dry state to a few ohms when the enclosure is filled with the disinfectant solution and the salt enclosed therein in dissolved in the disinfectant solution. In some embodiments, the threshold resistance at which the light source is activated may be in a range from about 10Ω to about 500Ω. For example, the threshold resistance may be about 10Ω, about 15Ω, about 20Ω, about 25Ω, about 30Ω, about 35Ω, about 40Ω, about 45Ω, about 50Ω, about 55Ω, about 60Ω, about 65Ω, about 70Ω, about 75Ω, about 80Ω, about 85Ω, about 90Ω, about 95Ω, about 100Ω, about 110Ω, about 120Ω, about 130Ω, about 140Ω, about 150Ω, about 160Ω, about 170Ω, about 180Ω, about 190Ω, about 200Ω, about 220Ω, about 240Ω, about 260Ω, about 280Ω, about 300Ω, about 325Ω, about 350Ω, about 375Ω, about 400Ω, about 450Ω, about 500Ω, about or any value between any two of these values.

In some embodiments, the diffusion control membrane is formed of a material such as, for example, polyethersulfone (PES), polystyrene, molybdenum disulfide, graphene, or a combination thereof. The particular material for the diffusion control membrane may be selected based on the particular disinfectant for which the device is to be used. Thus, any material with tailorable pore size, pore structure and pore density may be used to form the diffusion control membrane.

For example, an aqueous solution of isopropyl alcohol (IPA) is the most commonly used disinfectant. The molecular size of the disinfectant is about 0.4 nm. Thus, in order to allow IPA to diffuse through the diffusion control membrane should have a pore size of greater than 0.4 nm. On the other hand, the pore size should be smaller than the molecular size of the undissolved salt so as to prevent the undissolved salt from diffusing out of the enclosure 115. Thus, the specific pore size for the diffusion control membrane depends on the particular salt being enclosed within the enclosure 115. However, those of ordinary skill in the art, upon understanding the present disclosure, will readily appreciate that the devices and methods disclosed herein are not limited to the use of IPA solution as disinfectant and can be suitably modified for any disinfectant of choice. Some of the commonly disinfectants used for disinfecting components of medical infusion apparatus include, but are not limited to, IPA solution, chlorhexidine gluconate, povidone-iodine, iodophors, or a combination thereof.

In some embodiments, where the disinfectant of choice is a solution of two solvents (e.g., IPA and water), the salt type and amount of salt is selected to ensure that the salt dissolves in the disinfectant solution without disrupting the azeotropic nature of the disinfectant solution. Thus, if the disinfectant is, for example, IPA solution, any salt that is co-dissolvable IPA and water may be selected.

Without wishing to be bound by theory, where the disinfectant is a solution of two solvents, the selected salt should dissolve in both the solvents forming the disinfectant solution because if the salt dissolves in only one of the two solvents forming the disinfectant solution, the solvents may become layered, resulting in evaporation of only one of the solvents while leaving behind the other solvent. The solvent left behind may continue to dissolve the salt, thereby resulting in no decrease in electrical conductivity despite evaporation of one of the disinfectant solvents. Such a situation may result in continuous activation of the light source 110, and any indication that the disinfectant has evaporated cannot be obtained.

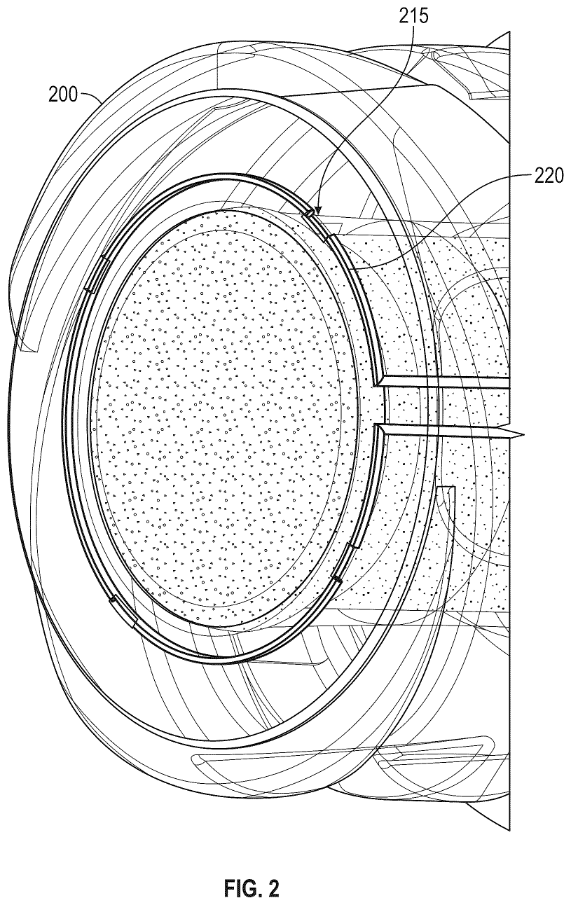

FIG. 2 shows a schematic of a needle-free connector with a device 100 embedded therein, in accordance with an example embodiment of the present disclosure. The needle-free connector 200 includes four (4) enclosures 215 disposed symmetrically around an opening thereof. The enclosures 215 are connected with lead wires 220.

The design parameters of the enclosure 215, the battery 105 and the light source 110 are based on the assumption that the connector 200 has a lifetime usage of about 200 times. Thus, if for example, it is assumed that the disinfectant solution evaporates in about 10 seconds, the light source 110 should remain activated (i.e., in ON state) for about 10 seconds for every use. This results in a total activation time of about 35-40 minutes over the lifetime of the connector.

Such needle-free connectors are typically used in a hospital setting with diffused indoor lighting. Thus, in order for a provider to suitably distinguish between the ON and OFF states of the light source 110, a minimum luminous intensity of the light source is desired to be about 10-20 mcd. For a typical light emitting diode (LED) that can emit light at an intensity in the range of about 10-20 mcd, the current usage during ON state, is in the range of about 50-100 mA.

Further, in order to suitably embed a battery in the needle-free connector, the battery size is restricted. Some commercially available batteries with form factors suitable to be embedded in a needle-free connector have a battery capacity in the range of about 400 mAh. Such a battery, at a discharge current of about 50-100 mA per activation, should last for about 4-8 hours of usage. In other words, a commercially available small-form factor battery would be sufficient to power the device 100 when embedded in a component such as the needle-free connector 200.

With these battery and light source parameters, in order to provide suitable resistance values, each of the enclosures 215 should have an “activation” resistance in the range of about 10 Ω to about 100Ω. In other words, the electrical resistance of the enclosure 215, when the salt is dissolved in the disinfectant solution should be in the range of about 10 to about 100Ω. To obtain such a resistance, within the design constraints of the ability to embed the device 100 into a component such as the needle-free connector 200, in one example, the enclosure may have a total volume of about 4 nl (about 400 μm×100 μm×100 μm) and enclose about 2 nl volume of CTAB/caprylic acid, leaving about 2 nl (about 15 ng by weight) for the disinfectant solution (70% v/v isopropyl alcohol solution in water). Such an enclosure, upon filling with the disinfectant solution is estimated to have an electrical resistance of about 50Ω (based on CTAB conductivity of about 700 μmS/cm).

In another aspect of the present disclosure, a method for disinfection of a component used in medical infusion tubing is provided. The method may include applying a disinfectant to the component until the light source is activated, and using the component when the light source is deactivated. The component includes, either via externally coupling or embedded therewithin, a device for providing confirmation of completion of disinfection such as, for example, device 100. Thus, in some embodiments, the method may further include coupling a confirmation device such as, for example, device 100, to the component prior to applying the disinfectant.

In some embodiments, applying the disinfectant may include scrubbing the component with a swab comprising the disinfectant. The swab comprising the disinfectant may be, for example, a swab soaked in the disinfectant solution. The disinfectant, in some embodiments, may be an aqueous solution of isopropyl alcohol or chlorhexidine.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A device for obtaining confirmation of completion of disinfection, the device comprising:
an electrically activated light source; and
at least one enclosure having at least one wall thereof comprising a diffusion control membrane and enclosing a salt, which is soluble in a disinfectant used for the disinfection,
wherein the at least one enclosure and the light source are electrically connected in series.

2. The device of claim 1, wherein the diffusion control membrane is configured to be permeable to the disinfectant and impermeable to undissolved salt.

3. The device of claim 1, wherein the diffusion control membrane is configured to control permeation of the disinfectant through the diffusion control membrane such that ionic mobility of the salt dissolved in the disinfectant is sufficient to enable activation of the light source only after the disinfectant has permeated through the diffusion control membrane for a predetermined amount of time.

4. The device of claim 3, wherein the predetermined amount of time is in a range from about 1 second to about 1 minute.

5. The device of claim 3, wherein after permeation of the disinfectant through the diffusion control membrane for the predetermined amount of time, electrical resistance of the enclosure reduces to be in a range from about 10 to about 100 Ω.

6. The device of claim 1, further comprising a power supply.

7. The device of claim 1, wherein the salt comprises an inorganic salt.

8. The device of claim 1, wherein the light source is a light emitting diode.

9. The device of claim 1, wherein the diffusion control membrane comprises a mate-rial selected from the group consisting polyethersulfone (PES), polystyrene, molybdenum disul-fide, and graphene.

10. A needle-free connector comprising:
a power supply;
an electrically activated light source; and
at least one enclosure having at least one wall thereof comprising a diffusion control membrane and enclosing a salt, which is soluble in a disinfectant used for disinfecting a surface of the needle-free connector to be disinfected, the at least one enclosure being disposed along the surface of the needle-free connector to be disinfected,
wherein the at least one enclosure, the power supply and the light source are electrically connected in series.

11. The needle-free connector of claim 10, wherein the diffusion control membrane is configured to control permeation of the disinfectant through the diffusion control membrane such that ionic mobility the salt dissolved in the disinfectant is sufficient to enable activation of the light source only after the disinfectant has permeated through the diffusion control membrane for a predetermined amount of time.

12. The needle-free connector of claim 11, wherein the predetermined amount of time is in a range from about 1 second to about 1 minute.

13. The needle-free connector of claim 11, wherein after permeation of the disinfect-ant through the diffusion control membrane for the predetermined amount of time, electrical resistance of the enclosure reduces to be in a range from about 10 to about 100 Ω.

14. The needle-free connector of claim 10, wherein the diffusion control membrane is configured to be permeable to the disinfectant and impermeable to undissolved salt.

15. The needle-free connector of claim 10, wherein the diffusion control membrane comprises a material selected from the group consisting polyethersulfone (PES), polystyrene, molybdenum disulfide, and graphene.

16. The needle-free connector of claim 10, wherein the salt comprises an inorganic salt selected from the group consisting of potassium chloride, sodium chloride, and magnesium chloride, or an organic salt selected from the group consisting of cetyltrimethylammonium bromide or caprylic acid.

17. The needle-free connector of claim 10, wherein the at least one enclosure has a volume in a range from about 1 nl to about 1 μl.

18. A method for disinfecting a needle-free connector, the method comprising:
applying a disinfectant to a needle-free connector having a confirmation device coupled thereto, the confirmation device comprising:
a power supply,
an electrically activated light source, and
at least one enclosure having at least one wall thereof comprising a diffusion control membrane and enclosing a salt, which is soluble in a disinfectant used for the disinfection, wherein the at least one enclosure, the power supply and the light source are electrically connect-ed in series, wherein the disinfectant is applied until the light source is activated; and
using the needle-free connector when the light source is deactivated.

19. The method of claim 18, wherein the diffusion control membrane is configured to control permeation of the disinfectant through the diffusion control membrane such that ionic mobility the salt dissolved in the disinfectant is sufficient to enable activation of the light source only after the disinfectant has permeated through the diffusion control membrane for a predetermined amount of time.

20. The method of claim 19, wherein the predetermined amount of time is in a range from about 1 second to about 1 minute.
